(19) United States
(12) Patent Application Publication
(10) Pub. No.: US 2006/0183935 A1
(43) Pub. Date: Aug. 17, 2006

(54) Processing improvements for hindered, ester-substituted phenols

(75) Inventors: Christopher M. Carter, Chardon, OH (US); Jason R. Brown, North Ridgeville, OH (US); Paul E. Adams, Willoughby Hills, OH (US)

Correspondence Address: Jason S. Fokens;The Lubrizol Corporation, Patent Dept./Mail Drop 022B, 29400 Lakeland Blvd., Wickliffe, OH, 44092-2298, US

(73) Assignee: The Lubrizol Corporation

(21) Appl. No.: 11/058,544

(22) Filed: Feb. 15, 2005

(51) Int. Cl. C07C 69/76

(52) U.S. Cl. 560/75

(57) ABSTRACT

The present invention related to a novel continuous process for the manufacture of hindered, ester-substituted phenols using 2,6-dialkylphenol a basic catalyst and acrylate esters.

### BACKGROUND OF THE INVENTION

The present invention relates to continuous production of hindered, ester-substituted phenols in continuously-stirred reactors.

It is known to use a hindered, ester-substituted phenol antioxidant in an oil of lubricating viscosity to reduce oxidation breakdown and improve cleanliness. These antioxidants are prepared by a number of processes.

Traditionally, hindered, ester-substituted phenols were made by an alkylation, reduction and transesterification process. This process generated high yields and a hindered, ester-substituted phenol product with a high degree of purity, which has a stabilizing effect on oxidizable organic materials when such materials are exposed to oxidative degradative conditions. However, this process requires elevated reaction temperatures, longer reaction times and generates by-products.

This manufacturing process is illustrated in U.S. Pat. No. 4,091,225, Parker, May 23, 1978, which discloses a method of making a hindered ester-substituted phenol by an alkylation, reduction and transesterification process to prepare materials of the general formula 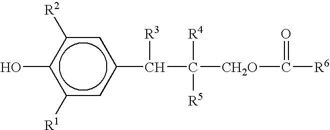
wherein R¹ and R² are the same or different radicals selected from the group consisting of tertiary alkyl radicals having from 4 to 12 carbon atoms, R³ is selected from the group consisting of hydrogen, methyl and ethyl, R⁴ and R⁵ are the same or different radicals selected from the group consisting of methyl, ethyl, n-propyl and n-butyl and R⁶ is selected from the group consisting of methyl, ethyl, propyl and butyl, aralykyl radicals having from 7 to 12 carbon atoms, vinyl, 2-propenyl and aryl radicals.

U.S. Pat. No. 4,659,863, Burton, Apr. 21, 1987, discloses a process which enhances conversion and speeds the reaction rate for the preparation of esters. Methyl acrylate and a hindered phenol are reacted (i) in the presence of an alkali metal alkaline catalyst that provides a phenolate anion and (ii) in the presence of an agent which solubilizes the phenolate to enhance the reaction rate.

U.S. Pat. No. 5,206,414, Evans et al., Apr. 27, 1993, discloses a process for the preparation of compounds of the general formula 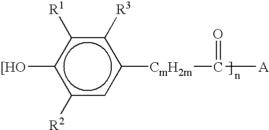
wherein R¹ and R² are identical or different and are hydrogen, C₁-C₁₈ alkyl, phenyl, C₁-C₄ alkyl-substituted phenyl, C₇-C₉ phenylalkyl, C₅-C₁₂ cycloalkyl or C₁-C₄ alkyl-substituted C₅-C₁₂ cycloalkyl, R³ is hydrogen or methyl, m is 0,1,2, or 3 and n is a number from 1 to 4 or 6, and A can be —OR⁴ where R⁴ can be C₂-C₄₅ alkyl.

U.S. Pat. No. 6,787,663, Adams et al., Sep. 7, 2004 discloses a process for the preparation of a hindered ester-substituted phenol and its use in a lubricant composition of the general formula 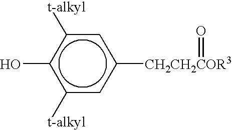
wherein R³ is an alkyl group containing 2 to 6 carbon atoms.

The present invention provides, among other advantages, a convenient method for obtaining a certain class of hindered phenolic ester antioxidants having particularly useful properties. In particular the antioxidants (described below) can be conveniently prepared in a series of continuous stirred tank reactors. In one synthesis, no transesterification reaction is necessary, resulting in a simplified process which utilizes lower reaction temperatures and shorter reaction times, which leads to fewer by-products. The antioxidants thus prepared impart excellent thermal and oxidative stability to lubricant formulations.

### SUMMARY OF THE INVENTION

The present invention provides a continuous process for preparing a hindered, ester-substituted phenol, comprising the steps of:

(a) heating a 2,6-dialkylphenol with a basic catalyst or mixture of basic catalysts in at least one reactor;

(b) continuously feeding the product of (a) and at least one acrylate ester to at least one continuous-stirred reactor;

(c) maintaining the mixture of (b) at a temperature of at least 130° C. for a residence time such that at least 80 percent by weight of the 2,6-dialkylphenol has reacted with the acrylate ester; thereafter,

(d) removing at least a portion of the basic catalyst from the reaction mixture;

thereby obtaining the hindered, ester-substituted phenol.

The invention further provides a method for lubricating an internal combustion engine, comprising supplying thereto a lubricant comprising the hindered, ester-substituted phenol as described herein.

### DETAILED DESCRIPTION OF THE INVENTION

Various preferred features and embodiments will be described below by way of non-limiting illustration.

Hindered ester-substituted phenol of the present invention can be of the type represented by the formula 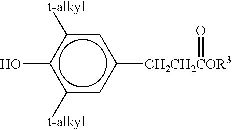
and in one embodiment

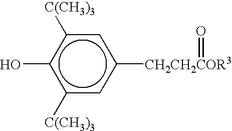

In these structures R³ can be a straight chain or branched chain alkyl group containing in certain embodiments 2 to 18 carbon atoms, or 2 to 12, or in another embodiment 2 to 10, or 2 to 8 or 6 carbon atoms, in yet another embodiment 4 carbon atoms. R³ is in one embodiment an n-butyl group.

Hindered, ester-substituted phenols of this type can be prepared by heating a 2,6-dialkylphenol with an acrylate ester under base catalysis conditions, such as aqueous KOH. This process is illustrated by the following example found in U.S. Pat. No. 6,787,663:

To a 5-L round-bottomed 4-necked flask, equipped with a mechanical stirrer, thermal probe, and reflux condenser equipped for distillate removal, is charged 2619 g 2,6-di-t-butylphenol and 17.7 g potassium hydroxide (technical grade). The reaction mixture is heated to 135° C. over 35 minutes and maintained at temperature for 2 hours, removing 9.7 g aqueous distillate. To the reaction mixture is charged 1466 g butyl acrylate dropwise over the course of 90 minutes. The temperature is maintained at 135° C. for up to 2 hours, or until analysis by infrared indicates no further change (by observing peaks at 727 and 768 cm⁻¹). To the mixture is charged 103 g magnesium silicate absorbent and 17 g filter aid and stirring is continued for 2 hours, while removing 7.1 g distillate. The mixture is filtered through additional filter aid.

While this process does not require a transesterification process, it is a batch process that does not have the conversion rates of the present invention's continuous process.

The present invention provides a method of preparing hindered ester-substituted phenols by the following process: (a) heating a 2,6-dialkylphenol with a basic catalyst or mixture of basic catalysts in at least one reactor; (b) continuously feeding the product of (a) and at least one acrylate ester to at least one continuous-stirred reactor; (c) maintaining the mixture of (b) at a temperature of at least 130° C. for a residence time such that at least 80 percent by weight of the 2,6-dialkylphenol has reacted with the acrylate ester; thereafter (d) removing at least a portion of the basic catalyst and of solids from the reaction mixture; thereby obtaining the hindered, ester-substituted phenol.

In one embodiment in step (a) the 2,6-dialkylphenol and basic catalyst or mixtures of basic catalyst is heated to a temperature of at least 100° C. to 300° C., in another embodiment to a temperature of at least 130° C. to 250° C. and in yet another embodiment to a temperature of at least 135° C. to 200° C., where these temperature is maintained.

The 2,6-dialkylphenol of step (a) can be 2,6-di-tert-butylphenol, 2,6-di-sec-butyl phenol, 2,6-di-iso-octyl phenol, or combinations thereof. The 2,6-dialklylphenol can be present in step (a) from 60 to 99.9 percent by weight of the total weight of basic catalyst and phenol; in another embodiment 70 to 99.7 percent by weight and in yet another embodiment 90 to 99.3 percent by weight. Specific illustrations can be 61 percent by weight as a function of total basic catalyst and 2,6-dialkylphenol, and 97 percent by weight as a function of total basic catalyst and 2,6-dialkylphenol.

The basic catalyst of step (a) can be potassium hydroxide, sodium hydroxide, cesium hydroxide or mixtures thereof. The basic catalyst can be in a solid or aqueous form. The aqueous form of the basic catalyst can contain, e.g., 25 to 55 percent by weight of water. Aqueous is defined as a water containing solution. The basic catalyst can be present in step (a) from 0.1 to 4.0 percent by weight of the total weight of basic catalyst and phenol; in another embodiment 0.2 to 3.5 percent by weight and in another embodiment 0.4 to 3.0 percent by weight and in yet another embodiment 0.6 to 2.0 percent by weight. Specific illustrations can be 3 percent by weight as a function of total basic catalyst and 2,6-dialkylphenol, and 0.6 percent by weight as a function of total basic catalyst and 2,6-dialkylphenol.

The reactor of step (a) can be a batch-wise reactor, continuous stirred tank reactor, semicontinuous reactor, tank reactor, tubular reactor, tower reactor or combination thereof.

The acrylate ester of step (b) can be butyl acrylate, methyl acrylate, ethyl acrylate, iso-octyl acrylate, 2-ethyl-hexyl acrylate, C₈-C₁₀ alkyl acrylate or mixtures thereof.

In one embodiment the acrylate ester of (b) is present in an amount of 20 to 55 percent by weight of the combined total weight of the components in step (a) and (b). In another embodiment the amount is 30 to 50 percent by weight and in yet another embodiment 40 to 45 percent by weight.

The process of step (b) can be accomplished in a series of continuous-stirred tank reactor (CSTR). In a CSTR the reactants are introduced and products with-drawn simultaneously in a continuous manner. A CSTR may assume the shape of a tank, a tubular structure, or a tower.

The mixture of step (b) can be maintained in the CSTR at a temperature of 100° C. to 300° C., in another embodiment 120° C. to 250° C., in another embodiment 130° C. to 200° C. for a residence time necessary to react 70 percent, in another embodiment 80 percent, in yet another embodiment 90 percent by weight of the 2,6-dialkylphenol in step (a) with the acrylate ester in step (b).

To calculate or determine the amount of the 2,6-dialkylphenol in step (a) that is reacted or converted, a Carbon 13 NMR can be utilized. A FTIR method can later be utilized to make this conversion determination by correlating the Carbon 13 NMR results to an IR ratio. The FTIR method ratios the net absorbance of a strong absorbance of the desired product at approximately 768 cm⁻¹ to a strong absorbance of the starting 2,6-di-t-butylphenol at approximately 746 cm⁻¹.

After the system has achieved steady state, the residence necessary to react the 2,6-dialkylphenol in step (a) with the acrylate ester in step (b) can be 1 to 10 hours, in another embodiment 2 to 8 hours, and in yet another embodiment 3 to 6 hours.

When the desired amount of the 2,6-dialkylphenol in step (a) has reacted with the acrylate ester in step (b) at least a portion of the basic catalyst and of any solids are removed thereby obtaining the hindered ester-substituted phenol as the remaining liquid. After the desired conversion in step (c) of the 2,6-dialkylphenol is attained, magnesium silicate adsorbent can be added to the mixture to aid in the removal, and allowed to remain in the batch for 1 hour or more to allow a portion of the basic catalyst to adsorb therein. In step (d) the batch can be then passed through a plate-and-frame filter (or other clarification device) to remove the solids (magnesium silicate and adsorbed catalyst-containing molecules). The batch can be filtered until the product contains no residual solids and no haze.

As used herein, the term “hydrocarbyl substituent” or “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include: hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form a ring); substituted hydrocarbon substituents, that is, substituents containing non-hydrocarbon groups which, in the context of this invention, do not alter the predominantly hydrocarbon nature of the substituent (e.g., halo (especially chloro and fluoro), hydroxy, alkoxy, mercapto, alkylmercapto, nitro, nitroso, and sulfoxy); hetero substituents, that is, substituents which, while having a predominantly hydrocarbon character, in the context of this invention, contain other than carbon in a ring or chain otherwise composed of carbon atoms. Heteroatoms include sulfur, oxygen, nitrogen, and encompass substituents as pyridyl, furyl, thienyl and imidazolyl. In general, no more than two, preferably no more than one, non-hydrocarbon substituent will be present for every ten carbon atoms in the hydrocarbyl group; typically, there will be no non-hydrocarbon substituents in the hydrocarbyl group.

It is known that some of the materials described above may interact in the final formulation, so that the components of the final formulation may be different from those that are initially added. For instance, metal ions (of, e.g., a detergent) can migrate to other acidic or anionic sites of other molecules. The products formed thereby, including the products formed upon employing the composition of the present invention in its intended use, may not be susceptible of easy description. Nevertheless, all such modifications and reaction products are included within the scope of the present invention; the present invention encompasses the composition prepared by admixing the components described above.

### EXAMPLES

The invention will be further illustrated by the following examples, which set forth particularly advantageous embodiments. While the Examples are provided to illustrate the present invention, they are not intended to limit it.

### Example 1

### Continuous Processing of Hindered Phenolic Ester with a Continuous Phenoxide Formation

Part 1: Continuous Phenoxide Precursor Formation Reactor System

The system consists of one or two continuous stirred tank reactors arranged in a series. Each reactor is approximately 45 L in size. A sub-line nitrogen flow is applied to all reactors at a rate equivalent to four tank turnovers per hour (9.0 kg of previously made phenoxide precursor can optionally be charged to the first reactor and heated to 146° C.). 2,6-di-tert-butyl phenol is fed to the first reactor at a rate of 2.24 kg/hr. Simultaneously, aqueous potassium hydroxide is fed to the first reactor at a rate of 0.026 kg/hr. The contents of the first reactor are maintained at a temperature of 146° C. Material of the first reactor can flow into the optional second reactor which provides additional space and reaction run time. The second reactor is set to overflow to a separate catch tank with an option to feed back into the first reactor. This overflow from the second reactor is the phenoxide precursor. The levels in each reactor are maintained to achieve a residence time of approximately 2-4 hours in each reactor once the system achieves steady state.

Part 2: Continuous Hindered Ester Substituted Phenol Formation System

The phenoxide precursor from Part 1 is directed to the first of a series of four 45 L continuous stirred tank reactors at a rate of 2.25 kg/hr (9.1 kg of untreated/unfiltered hindered, ester-substituted phenol can optionally be charged to the first reactor of the system and heated to 141° C.). Simultaneously, n-butyl acrylate is fed into the first reactor at a rate of 1.65 kg/hr. The resulting contents of this first reactor are maintained at 141° C. and the contents of the remaining reactors are maintained at 135° C. The contents of the first reactor will overflow into the second reactor and thereafter into the subsequent reactors. The overflow of the fourth reactor, which is hindered ester-substituted phenol, is sent to a separate catch tank. The levels in each reactor are maintained to achieve a residence time of approximately 2.25 hours in each reactor once the system is at steady state.

The effluent and/or overflow from the continuous reaction process, which is hindered ester substituted phenol, is further processed by passing through a continuous flash stripper to remove the residual n-butyl acrylate to a level at or below 200 ppm by weight. The material is further processed by treating with magnesium silicate in an amount as required to reduce the residual potassium after filtration to 400 ppm potassium by weight or less. The magnesium silicate and other processing solids are removed by filtration.

### Example 2

### Continuous Processing of Hindered Phenolic Ester with a Continuous Phenoxide Formation using a Mixed Catalyzed Process (Potassium Hydroxide and Sodium Hydroxide)

The same procedure of Example 1 is used except that in the continuous phenoxide precursor formation reactor system of part 1, aqueous potassium hydroxide at a rate of 0.019 kg/hr and aqueous sodium hydroxide at a rate of 0.013 kg/hr are fed simultaneously into the first reactor.

### Example 3

### Continuous Processing of Hindered Phenolic Esters (Mixture of n-butyl and Methyl Acrylate) with a Continuous Phenoxide Formation

The same procedure of Example 1 is used except that in the four-reactor system of part two, n-butyl acrylate is fed into the first reactor at a rate of 1.52 kg/hr and at simultaneously, methyl acrylate is fed into the same reactor at a rate of 0.08 kg/hr.

The use of a mixture of n-butyl and methyl acrylate in the process can preclude crystallation of the hindered ester-substituted phenol.

### Example 4

### Continuous Processing of Hindered Phenolic Esters (Mixture of n-butyl and 2-ethylhexyl Acrylate) with a Continuous Phenoxide Formation

The procedure of Example 1 is used except that in the four-reactor system of part two, n-butyl acrylate is fed into the first reactor at a rate of 1.32 kg/hr and simultaneously 2-ethylhexyl acrylate is fed into the same reactor at a rate of 0.47 kg/hr.

Each of the documents referred to above is incorporated herein by reference. Except in the Examples, or where otherwise explicitly indicated, all numerical quantities in this description specifying amounts of materials, reaction conditions, molecular weights, number of carbon atoms, and the like, are to be understood as modified by the word “about.” Unless otherwise indicated, each chemical or composition referred to herein should be interpreted as being a commercial grade material which may contain the isomers, by-products, derivatives, and other such materials which are normally understood to be present in the commercial grade. However, the amount of each chemical component is presented exclusive of any solvent or diluent oil, which may be customarily present in the commercial material, unless otherwise indicated. It is to be understood that the upper and lower amount, range, and ratio limits set forth herein may be independently combined. Similarly, the ranges and amounts for each element of the invention can be used together with ranges or amounts for any of the other elements. As used herein, the expression “consisting essentially of” permits the inclusion of substances that do not materially affect the basic and novel characteristics of the composition under consideration.

What is claimed is:

1. A continuous process using two or more vessels for preparing a hindered, ester-substituted phenol, comprising the steps of:
(a) heating a 2,6-dialkylphenol with a basic catalyst or mixture of basic catalysts in at least one vessel;
(b) continuously feeding the product of (a) and at least one acrylate ester to at least one continuously-stirred vessel;
(c) maintaining the mixture of (b) at a temperature of at least 130° C. for a residence time such that at least 80 percent by weight of the 2,6-dialkylphenol has reacted with the acrylate ester; and thereafter
(d) removing at least a portion of the basic catalyst from the reaction mixture;
thereby obtaining the hindered ester-substituted phenol.

2. The process of claim 1 wherein the basic catalyst comprises potassium hydroxide, sodium hydroxide, cesium hydroxide or mixtures thereof in aqueous or solid form.

3. The process of claim 1 wherein the basic catalyst is in aqueous form and the vessel to which the basic catalyst is added is maintained at a temperature of at least 100° C.

4. The process of claim 1 wherein the acrylate ester comprises butyl acrylate, methyl acrylate, ethyl acrylate, iso-octyl acrylate, 2-ethyl-hexyl acrylate, C8-C10 alkyl acrylate or mixtures thereof.

5. The process of claim 4 wherein the acrylate ester comprises a mixture of butyl acrylate and methyl acrylate.

6. The process of claim 1 wherein at least two vessels in series are present in step (a) and at least a portion of the 2,6-dialkylphenol or basic catalyst is added to a vessel other than the first vessel of step (a).

7. The process of claim 1 wherein at least two vessels in series are present in step (b) and a portion of the acrylate ester is added to a vessel other than the first vessel of step (b).

8. The process of claim 1 wherein the 2,6-dialkylphenol is 2,6-di-tert-butylphenol.

9. The process of claim 1 wherein step (a) is conducted in a batch-wise vessel.

10. The process of claim 1 wherein step (a) is conducted in a continuously-stirred vessel.

11. The process of claim 1 wherein the basic catalyst is removed by treatment with magnesium silicate.

12. The process of claim 1 wherein the magnesium silicate is stirred with the reaction mixture and subsequently filtered.

13. The process of claim 1 wherein the mixture is filtered using a filter aid.

14. A continuous process using two or more vessels for preparing a hindered, ester-substituted phenol, comprising the steps of:
(a) heating a 2,6-dialkylphenol with aqueous potassium hydroxide, sodium hydroxide, cesium hydroxide or mixtures thereof in at least one continuous-stirred vessel;
(b) continuously feeding the product of step (a) and methyl acrylate, butyl acrylate or mixtures thereof to at least one continuously-stirred vessel;
(c) maintaining the mixture of (b) at a temperature of at least 130° C. for a residence time such that at least 80 percent by weight of the 2,6-dialkylphenol has reacted with the acrylate ester; and thereafter
(d) removing at least a portion of the basic catalyst from the reaction mixture;
thereby obtaining the hindered ester-substituted phenol.

15. The product prepared by the process of claim 1.
